(19) United States
(12) Patent Application Publication
(10) Pub. No.: US 2011/0020408 A1
(43) Pub. Date: Jan. 27, 2011

(54) MULTILAYERED MODIFIED RELEASE FORMULATION COMPRISING AMOXICILLIN AND CLAVULANATE

(75) Inventors: Kalaiselvan Ramaraju, Tamil Nadu (IN); Rajan Kumar Verma, New Delhi (IN); Ashok Rampal, Maharashtra (IN)

Correspondence Address: Ranbaxy Inc., Intellectual Property Department, 600 College Road East, PRINCETON, NJ, 08540, US

(73) Assignee: RANBAXY LABORATORIES LIMITED, Gurgaon (IN)

(21) Appl. No.: 12/600,590

(22) PCT Filed: May 16, 2008
(86) PCT No.: PCT/IB08/51944

§ 371 Date: Sep. 17, 2010

(30) Foreign Application Priority Data
May 17, 2007 (IN) 1074/DEL2007

(51) Int. Cl. A61K 9/00; A61K 31/43; A61P 31/00

(52) U.S. Cl. 424/400; 514/197

(57) ABSTRACT

The present invention relates to multilayered modified release formulation comprising amoxicillin and clavulanate, process of preparation thereof and method of treating bacterial infection using these formulations. The multilayered modified release formulation comprises: an immediate release layer comprising amoxicillin and clavulanate; and a slow release layer comprising amoxicillin and one or more release retarding agents; and one or more non-release controlling inert barrier layers placed in between the immediate release layer and the slow release layer and comprising one or more pharmaceutically acceptable excipients.

### FIELD OF THE INVENTION

The present invention relates to a multilayered modified release formulation comprising amoxicillin and clavulanate, process of preparation thereof and method of treating bacterial infection using these formulations.

### BACKGROUND OF THE INVENTION

Amoxicillin is an analog of ampicillin, derived from the basic penicillin nucleus 6-aminopenicillanic acid. Chemically, amoxicillin is (2S,5R,6R)-6-[(R)-(−)-2-amino-2-(p-hydroxyphenyl)acetamido]-3,3-dimethyl-7-oxo-4-thia-1-azabicyclo[3.2.0]heptane-2-carboxylic acid.

Clavulanic acid is produced by the fermentation of Streptomyces clavuligerus. It is a β-lactam structurally related to penicillins and possesses the ability to inactivate a wide variety of β-lactamases by blocking the active sites of these enzymes. Clavulanic acid is particularly active against the clinically important plasmid-mediated β-lactamases frequently responsible for transferred drug resistance to penicillins and cephalosporins. Chemically, clavulanic acid is (Z)-(2R,5R)-3-(2-hydroxyethylidene)-7-oxo-4-oxa-1-azabicyclo[3.2.0]-heptane-2-carboxylate. Clavulanic acid is highly prone to degradation in the presence of moisture.

The pharmaceutical formulations comprising amoxicillin and clavulanate approved for marketing in U.S. are immediate release and modified release formulations. These formulations contain various weight ratios of amoxicillin and potassium clavulanate ranging from 2:1 to 16:1. For instance, conventional swallow tablets comprising 250/125, 500/125, 500/62.5, and 875/125 mg amoxicillin/clavulanic acid (in the form of potassium clavulanate). Such tablets comprise amoxicillin and clavulanic acid in the ratio 2:1, 4:1, 8:1 and 7:1, respectively. The 875/125 mg tablet was developed to provide a tablet formulation, which could be administered in a bid (twice daily) dosage regimen. It is also marketed for tid (three times daily) dosing, in Italy and Spain. The 500/62.5 mg tablet was also developed to provide a tablet formulation, which could be administered in a bid dosage regimen, two such tablets being taken every 12 hours, in preference to a single 1000/125 mg tablet. A 1000/125 mg single dosage is also available, in France, but as a single dosage sachet rather than a tablet. Typically, the approved regimen provides a single dosage of 125 mg of potassium clavulanate. Augmentin-XR® containing 1000 mg of amoxicillin and 62.5 mg of clavulanic acid, is available in U.S. The recommended dose of Augmentin-XR® is 4,000 mg/250 mg daily (ie. 2 tablets of 1000/62.5 mg twice daily).

It is well known in the pharmaceutical art to prepare formulations, which provide for modified release of pharmacologically active substances after oral administration to humans and animals. Multilayered tablets provides us with the possibility of obtaining products capable of releasing one or more drugs at different rates or else releasing the two drugs sequentially.

Various multilayered modified release tablets are previously known in the art.

U.S. Pat. No. 6,372,255, assigned to Merck Patent Gesellschaft, describes a multilayer tablet comprising at least two superposed layers, wherein; a first outer layer comprises a mixture of excipients and a first active substance, wherein the first layer allows immediate release of the first active substance; and a second layer, which is in contact with the first layer, comprises at least one non-biodegradable, inert porous polymeric matrix in which a second active substance is dispersed; allowing for the prolonged release of the second active substance.

U.S. Pat. No. 6,294,200, assigned to Jagotec AG, teaches a tablet comprising a three-layered core covered by a partial coating layer, the core having an upper layer consisting of active substance and suitable excipients to allow a fast release of the active substance when the tablet comes into contact with an aqueous medium; an intermediate layer comprising polymeric material suitable to form a barrier able to determine a time interval between the release of the active substance contained in the upper layer and the active substance contained in the lower layer, a lower layer comprising one or more active substances and having the same or a different composition as the upper layer, the lower layer allowing the controlled release of the active substances; and wherein the partial coating layer consists of granulated polymeric substances, adjuvant substances and plasticizing agents applied by compression on the whole lateral surface and on the lower base of the three layered core thus forming an impermeable barrier which resists dissolution for a predetermined period of time while allowing for the release of the active substance both from the upper layer and from the lower layer.

WO 94/06416, assigned to Jagotec AG, teaches a tablet comprising a three-layered core comprising first layer containing one or more drugs, a second layer containing one or more drugs and a low-permeability barrier type layer placed between the first and the second layer in order to offer the advantage of releasing the drug or drugs according to a prefixed schedule. The low-permeability barrier type layer is impermeable to the drug of the adjacent layer for at least 4-6 hours.

WO 03/101431, assigned to J. B. Chemicals and Pharmaceuticals Ltd., teaches a tablet containing two or more layers, at least one layer for immediate delivery of an active agent, second layer for controlled delivery of an active agent that includes a gas generating agent, gelling agent and optionally a third layer placed in between first and second layer comprising inert excipients to facilitate the delivery of two incompatible active agents. The tablet floats in the fluid of the environment (e.g., the stomach), thereby being retained in the environment of use for an extended period of time.

WO 95/20946, assigned to Smithkline Beecham, teaches multilayered tablet formulations comprising a first layer which includes amoxicillin and/or clavulanate, a second layer which includes amoxicillin and/or clavulanate, wherein the relative rate of release of amoxicillin and/or clavulanate from the first and second layers differs. The tablet formulation may optionally include a barrier layer that is either substantially or completely impermeable to aqueous media or is slowly erodable in aqueous media.

WO 00/61115, assigned to Smithkline Beecham, discloses a modified release pharmaceutical formulation comprising 1000 mg amoxicillin and 62.5 mg potassium clavulanate in which all of the potassium clavulanate and a first part of amoxicillin are formulated with pharmaceutically acceptable excipients which allow for immediate release of the potassium clavulanate and the first part of amoxicillin, to form an immediate release phase, and further comprising a second part of amoxicillin formulated with pharmaceutically acceptable excipients which allow for slow release of the second part of amoxicillin, to form a slow release phase. The slow release phase comprises a release retarding excipient. Xanthan gum and organic acid are the preferable release retarding excipients. The patent teaches that the tablet formulation may also include one or more barrier layers, which may be located between the first and second layers and such barrier layers are composed of polymers which are either substantially or completely impermeable to water or aqueous media or are slowly erodable in water or aqueous media or biological liquids and/or which swell in contact with water or aqueous media. The barrier layer retains its characteristics at least until complete or substantially complete transfer of the active material content.

The above-cited prior art references teach the use of barrier layers (placed in between the first and second layers containing the active agents) to provide a time gap, i.e., to release the active agent according to prefixed schedule, by incorporating polymers which are either substantially or completely impermeable to water or aqueous media or are slowly erodable in water or aqueous media or biological liquids and/or which swell in contact with water or aqueous media.

However, the prior art does not envisage the possibility of using these barrier layers to increase the release of certain drugs such as clavulanic acid or its salts thereof. Swellable and gellable polymers such as hydroxypropyl methylcellulose, methyl cellulose, carboxy methyl cellulose, gums, polethylene oxide, carbomer etc. are among the first choice polymers for controlling the release. These polymers have very strong tendency to gel after coming in contact with water. It was observed that bilayer tablets with such polymers in the slow release layer causes clavulanic acid or salts thereof in the immediate release layer to get stuck to the slow release layer because of gel formation, as a result of which incomplete/slow release of clavulanic acid was observed. Since clavulanic acid degrades in presence of moisture, appreciable degradation of clavulanic acid occurs in GIT before absorption can take place thereby resulting in lower bioavailability. It has surprisingly been found that the incorporation of an inert barrier layer between the immediate and slow release layers facilitates the release of clavulanic acid. Unlike prior art formulations, this barrier layer does not impede the release of the active agents from the adjacent layers. Thus, the present invention relates to a multilayered modified release formulation of amoxicillin and clavulanate comprising an immediate release layer, a slow release layer and one or more non-release controlling inert barrier layers placed in between the immediate release layer and the slow release layer. The modified release formulation of the present invention comprises amoxicillin and clavulanate in a weight ratio of 16:1.

### SUMMARY OF THE INVENTION

According to one embodiment there is provided a multilayered modified release formulation comprising:
an immediate release layer comprising amoxicillin and clavulanate,
a slow release layer comprising amoxicillin and one or more release retarding agents; and,
one or more non-release controlling inert barrier layers placed in between the immediate release layer and the slow release layer and comprising one or more pharmaceutically acceptable excipients.

The formulation can contain amoxicillin and clavulanate in a ratio of about 16:1.

According to another embodiment there is provided a multilayered modified release formulation which comprises 1000 mg±5% of amoxicillin and 62.5 mg±5% of clavulanate, comprising:
an immediate release layer comprising amoxicillin and clavulanate,
a slow release layer comprising amoxicillin and one or more release retarding agents; and,
one or more non-release controlling inert barrier layers placed in between the immediate release layer and the slow release layer and comprising one or more pharmaceutically acceptable excipients.

According to another embodiment there is provided a multilayered modified release formulation which comprises 1000 mg±5% of amoxicillin and 62.5 mg±5% of clavulanate, comprising:
an immediate release layer comprising amoxicillin and clavulanate,
a slow release layer comprising amoxicillin and 0.5-50% w/w of one or more release retarding agents based on the weight of the slow release layer; and,
one or more non-release controlling inert barrier layers placed in between the immediate release layer and the slow release layer and comprising one or more pharmaceutically acceptable excipients.

According to one more embodiment, the multilayered modified release formulation of the invention releases more than 50% of clavulanic acid within 15 minutes and more than 40% of amoxicillin within 30 minutes, when in vitro release profile is measured using USP-2 method, at 50 rpm, in 900 ml water at 37±0.5° C.

According to another embodiment, there is provided a process for preparing the multilayered modified release formulations disclosed in the various embodiments of the specification.

### DETAILED DESCRIPTION OF THE INVENTION

The multilayered modified release formulation of the present invention comprises amoxicillin and clavulanate in a weight ratio of about 14:1 to about 20:1, for example, about 16:1. The amount of amoxicillin may range from about 1000 mg to about 2000 mg and the amount of clavulanate may range from about 62.5 mg to about 125 mg. For example, the amount of amoxicillin is 1000 mg±5% and the amount of clavulanate is 62.5 mg±5%.

The term “amoxicillin” as used herein, refers to amoxicillin, its alkali, alkaline, or acid salts, hydrates, solvates and mixtures thereof. For example, amoxicillin may be in the form of amoxicillin trihydrate or amoxicillin sodium and the clavulanate may be in the form of potassium clavulanate.

Unless otherwise indicated, weights of amoxicillin and potassium clavulanate (“clavulanate”) refer to the equivalent weights of the corresponding free acids. In addition, it will be appreciated that in practice, weights of amoxicillin and clavulanate to be incorporated into a formulation will be further adjusted, in accord with conventional practice, to take account of the potency of the amoxicillin and clavulanate.

As used herein, the term “immediate release” shall mean the release of the majority of the active material content within a relatively short time, for example within 1 hour or within 30 minutes after oral ingestion. Examples of such immediate release formulations include conventional swallow tablets, dispersible tablets, chewable tablets, single dose sachets and capsules, or effervescent forms thereof.

As used herein, the term “modified release” refers to the release of a drug substance from a pharmaceutical formulation, at a slower rate than from an immediate release formulation. For example, the modified release formulation includes both an immediate release phase and a slow release phase. Modified release formulations are well known in the art, see for instance Remington: The Science and Practice of Pharmacy, Nineteenth Edn, 1995, Mack Publishing Co., Pennsylvania, USA.

The multilayered modified release formulation according to present invention may be in the form of tablet, for example, a trilayered tablet.

The immediate release layer optionally comprises other pharmaceutically acceptable excipients. The slow release layer comprises one or more release retarding polymers and optionally other pharmaceutically acceptable excipients.

As used herein the term ‘non-release controlling inert barrier layer’ shall mean a layer that is non-active and which does not impede the release of amoxicillin or clavulanate from the adjacent layers. The inert barrier layer prevents the contact of the clavulanate containing immediate release layer with the slow release layer. The non-release controlling inert barrier layer comprises one or more pharmaceutically acceptable excipients.

The ‘release retarding polymer’ may be selected from one or more of gums, or hydrophilic polymers. The gums may be xanthan gum, guar gum, agar, carrageenan, tragacanth or acacia. The hydrophilic polymer may be cellulose derivatives, polyvinylalcohol, polyvinylpyrrolidone, polyethylene oxide, alginic acid or salts thereof. The cellulose derivatives used as hydrophilic polymer may be carboxymethylcellulose, hydroxypropyl cellulose or hydroxypropyl methylcellulose (for eg. the product used under the trade names Methocel K4MCR®, Methocel ES®)).

The ‘pharmaceutically acceptable excipients’ may be selected from one or more of fillers/diluents, disintegrants, binders, pH modifiers, lubricant/glidants and coloring agents.

The ‘fillers/diluents’ may be selected from one or more of microcrystalline cellulose, lactose, mannitol or calcium phosphate.

The ‘disintegrants’ may be selected from one or more of croscarmellose sodium, sodium starch glycolate, crospovidone, hydroxypropyl cellulose, pregelatinised starch, microcrystalline cellulose or mixtures thereof.

The ‘binders’ may be selected from one or more of polyvinylpyrrolidone, pregelatinised starch, methacrylic acid polymers, e.g., Eudragit E 100®, gelatin or hydroxypropyl cellulose.

The ‘pH modifiers’ may be selected from one or more of citric acid, ascorbic acid, tartaric acid, malic acid, malonic acid, succinic acid, fumaric acid, maleic acid, adipic acid, lactic acid, levulinic acid, sorbic acid, polyacrylic acid (for e.g., the product used under the trade names Carbopol 934P®), orthophosphoric acid, hydrochloric acid, nitric acid, sulphuric acid, sulfamic acid, hydrofluoric acid, oxoacids, sodium carbonate, sodium bicarbonate, magnesium carbonate, magnesium oxide, calcium carbonate, calcium oxide, aluminium hydroxide, magnesium hydroxide, sodium hydroxide or pharmaceutically acceptable salts thereof. For example, the pH modifier is sodium dihydrogen phosphate and/or potassium dihydrogen phosphate.

The lubricants/glidants' may be selected from one or more of talc, colloidal silicon dioxide, magnesium stearate or zinc stearate.

The modified release formulation may be prepared by wet granulation, dry granulation or direct compression process. The wet granulation process involves use of water or any other suitable solvent with or without binders. The dry granulation may involve use of roller compacter or any suitable technique. The preparation of layered tablets may involve preparing immediate release granules, non-release controlling inert barrier granules, and slow release granules. Such granules can then be formulated respectively into immediate release layer, non-release controlling inert barrier layer, and slow release layer.

According to one embodiment of the specification there is provided a process for the preparation of a multilayered modified release formulation, the process comprising the steps of:
a) preparing immediate release granules comprising amoxicillin, clavulanate and optionally one or more pharmaceutically acceptable excipients; and,
b) preparing non-release controlling inert barrier granules comprising one or more pharmaceutically acceptable excipients; and,
c) preparing slow release granules comprising amoxicillin, one or more release retarding agents and optionally one or more pharmaceutically acceptable excipients; and,
d) compressing the granules of steps (a), (b) and (c) into three separate layers.

A non-functional coating layer may optionally cover the modified release formulation. The coating layer may comprise polymers like hydroxypropyl cellulose, hydroxyethyl cellulose or hydroxypropyl methylcellulose; plasticisers like polyethylene glycol, triacetin, dibutyl sebecate or diethyl tartrate; opacifying agents like titanium dioxide or talc; and colouring agents.

The multilayered modified release formulation of the invention releases more than 50% of clavulanic acid within 15 minutes and more than 40% of amoxicillin within 30 minutes, when in vitro release profile is measured using USP-2 method, at 50 rpm, in 900 ml water at 37±0.5° C.

The modified release formulations of the various embodiments are described below in more detail without limiting the scope of the present invention:

### Comparative Example

| Ingredients | mg/tablet |
|---|---|
| A. Immediate Release Blend | |
| 1. Intragranular | |
| Potassium clavulanate + microcrystalline cellulose (1:1) | 152.44 |
| Amoxicillin trihydrate | 516.53 |
| Mannitol | 144.53 |
| Magnesium stearate | 4.00 |
| Colloidal silicon dioxide | 2.50 |
| Total | 820.00 |
| 2. Extragranular | |
| Mannitol | 16.00 |
| Sodium starch glycolate | 34.00 |
| Magnesium stearate | 5.00 |
| Colloidal silicon dioxide | 2.50 |
| Total | 877.50 |
| B. Slow Release Blend | |
| 1. Intragranular | |
| Amoxicillin trihydrate | 129.13 |
| Amoxicillin sodium | 463.82 |
| Microcrystalline cellulose | 50.07 |
| Hydroxypropyl methylcellulose (Methocel K4MCR ®) | 50.00 |
| Polyethylene oxide | 20.00 |
| Sodium dihydrogen phosphate anhydrous | 23.48 |
| Potassium dihydrogen phosphate anhydrous | 6.00 |
| Magnesium stearate | 5.00 |
| Total | 747.50 |
| 2. Extragranular | |
| Magnesium stearate | 10.00 |
| Colloidal silicon dioxide | 2.50 |
| Total | 760.00 |
| Total (A + B) | 1637.50 |

### Brief Manufacturing Procedure—

### Immediate Release Blend:

1. All the excipients were dried (except potassium clavulanate) to get equilibrium relative humidity (ERH) levels of not more than 10%.
2. The intragranular materials (potassium clavulanate, amoxicillin trihydrate, mannitol, magnesium stearate, and colloidal silicon dioxide) were blended and compacted.
3. The compacts of step 2 were milled and sifted to obtain granules.
4. The granules obtained in step 3 were blended with the extragranular excipients (mannitol, sodium starch glycolate, magnesium stearate, and colloidal silicon dioxide).

Slow Release Blend:
5. The intragranular excipients (amoxicillin trihydrate, amoxicillin sodium, microcrystalline cellulose, hydroxypropyl methylcellulose, polyethylene oxide, sodium dihydrogen phosphate, potassium dihydrogen phosphate, and magnesium stearate) were blended.
6. The blend of step 5 was compacted.
7. The compacts of step 6 were milled and sifted to obtain granules.
8. The granules of step 7 were dried to achieve granule's ERH level of NMT 10%.
9. The granules of step 8 were blended with extragranular excipients (magnesium stearate and colloidal silicon dioxide) pre-dried to ERH levels of not more than Bilayer Tablet Compression:
10. The weighed quantity of immediate release blend of step 4 was filled in the die and the blend was pre-compressed. Then slow release blend of step 9 was filled and final compression was done.

### Examples 1-4

| | mg/tablet | | | |
|---|---|---|---|---|
| Ingredients | Example 1 | Example 2 | Example 3 | Example 4 |
| A. Immediate Release | | | | |
| Blend | | | | |
| 1. Intragranular | | | | |
| Potassium clavulanate + | 152.44 | 152.44 | 152.44 | 152.44 |
| Microcrystalline | | | | |
| cellulose (1:1) | | | | |
| Amoxicillin trihydrate | 516.53 | 516.53 | 516.53 | 516.53 |
| Mannitol | 144.53 | 144.53 | 144.53 | 144.53 |
| Magnesium stearate | 4.00 | 4.00 | 4.00 | 4.00 |
| Colloidal silicon dioxide | 2.50 | 2.50 | 2.50 | 2.50 |
| Total | 820.00 | 820.00 | 820.00 | 820.00 |
| 2. Extragranular | | | | |
| Mannitol | 16.00 | 16.00 | 16.00 | 16.00 |
| Sodium starch glycolate | 34.00 | 34.00 | 34.00 | 34.00 |
| Magnesium stearate | 5.00 | 5.00 | 5.00 | 5.00 |
| Colloidal silicon dioxide | 2.50 | 2.50 | 2.50 | 2.50 |
| Total | 877.50 | 877.50 | 877.50 | 877.50 |
| B. Inert barrier blend | | | | |
| Microcrystalline cellulose | 145.5 | 147.0 | 73.5 | — |
| Lactose anhydrous | — | — | 73.5 | 147.0 |
| Sodium starch glycolate | 3.0 | — | — | — |
| Magnesium stearate | — | 1.5 | 1.5 | 1.5 |
| Iron oxide (red) | 1.5 | 1.5 | 1.5 | 1.5 |
| Total | 150 | 150 | 150 | 150 |
| Total (A + B) | 1027.50 | 1027.50 | 1027.50 | 1027.50 |
| C. Slow Release Blend | | | | |
| 1. Intragranular | | | | |
| Amoxicillin trihydrate | 129.13 | 129.13 | 129.13 | 129.13 |
| Amoxicillin sodium | 463.82 | 463.82 | 463.82 | 463.82 |
| Microcrystalline cellulose | 50.07 | 50.07 | 50.07 | 50.07 |
| Hydroxypropyl | 50.00 | 50.00 | 50.00 | 50.00 |
| methylcellulose | | | | |
| (Methocel K4MCR) | | | | |
| Polyethylene oxide | 20.00 | 20.00 | 20.00 | 20.00 |
| Sodium dihydrogen | 23.48 | 23.48 | 23.48 | 23.48 |
| phosphate anhydrous | | | | |
| Potassium dihydrogen | 6.00 | 6.00 | 6.00 | 6.00 |
| phosphate anhydrous | | | | |
| Magnesium stearate | 5.00 | 5.00 | 5.00 | 5.00 |
| Total | 747.50 | 747.50 | 747.50 | 747.50 |
| 2. Extragranular | | | | |
| Magnesium stearate | 10.00 | 10.00 | 10.00 | 10.00 |
| Colloidal silicon dioxide | 2.50 | 2.50 | 2.50 | 2.50 |
| Total | 760.00 | 760.00 | 760.00 | 760.00 |
| Total (A + B + C) | 1787.50 | 1787.50 | 1787.50 | 1787.50 |

### Brief Manufacturing Procedure

### Immediate Release Blend:

1. All the excipients were dried (except potassium clavulanate) to get equilibrium relative humidity (ERH) levels of not more than 10%.
2. The intragranular materials (potassium clavulanate, amoxicillin trihydrate, mannitol, magnesium stearate, and colloidal silicon dioxide) were blended and compacted.
3. The compacts of step 2 were milled and sifted to obtain granules.
4. The granules obtained in step 3 were blended with the extragranular excipients (mannitol, sodium starch glycolate, magnesium stearate, and colloidal silicon dioxide).

### Inert Barrier Blend:

5. All the ingredients were passed through BSS#60 and uniformly blended.

### Slow Release Blend:

6. The intragranular excipients (amoxicillin trihydrate, amoxicillin sodium, microcrystalline cellulose, hydroxypropyl methylcellulose, polyethylene oxide, sodium dihydrogen phosphate anhydrous, potassium dihydrogen phosphate anhydrous, and magnesium stearate) were blended.
7. The blend of step 6 was compacted.
8. The compacts of step 7 were milled and sifted to obtain granules.
9. The granules of step 8 were dried to achieve granule's ERH level of NMT 10%.
9. The granules of step 9 were blended with extragranular excipients (magnesium stearate and colloidal silicon dioxide) pre-dried to ERH levels of not more than 10%.

### Trilayer Tablet Compression:

10. The weighed quantity of immediate release blend of step 4 was filled in the die and the blend was pre-compressed. The inert barrier blend of step 5 was then filled and again pre-compression was done. Finally, the extended release blend of step 10 was filled and final compression was performed.

The in vitro release profile of clavulanic acid from formulations given in Comparative Example and Examples 1-4, as measured by the USP-2 method in 900 ml water at 50 rpm, is listed below:

| | Percent Dissolved of Clavulanic Acid | | | | |
|---|---|---|---|---|---|
| Time | Comparative | | | | |
| (min) | Example | Example 1 | Example 2 | Example 3 | Example 4 |
| 15 | 15 | 81 | 57 | 72 | 62 |
| 30 | 42 | 100 | 81 | 102 | 87 |
| 45 | 64 | 100 | 93 | 104 | 93 |
| 60 | 78 | 101 | 97 | 104 | 96 |
| 120 | 93 | 100 | 100 | 104 | 100 |

The in vitro release profile of amoxicillin from formulations given in Comparative

Example and Examples 1-4, as measured by the USP-2 method in 900 ml distilled water at 50 rpm, is listed below:

| | Percent Dissolved of Amoxicillin | | | | |
|---|---|---|---|---|---|
| Time | Comparative | | | | |
| (h) | Example | Example 1 | Example 2 | Example 3 | Example 4 |
| 0.5 | 31 | 53 | 45 | 53 | 48 |
| 1 | 51 | 57 | 56 | 59 | 57 |
| 2 | 66 | 63 | 65 | 66 | 67 |
| 3 | 73 | 68 | 72 | 71 | 75 |
| 4 | 79 | 72 | 77 | 76 | 79 |
| 5 | 83 | 75 | 80 | 80 | 81 |
| 6 | 85 | 78 | 83 | 82 | 84 |
| 8 | 88 | 82 | 85 | 85 | 85 |

As observed from the data, the release of clavulanic acid is faster from the tablets containing a non-release controlling barrier layer placed in between the clavulanate containing immediate release layer and the slow release layer when compared to the tablets without said barrier layer. Further, the release of amoxicillin from the slow release layer is not affected by the barrier layer.

We claim:

1. A multilayered modified release pharmaceutical formulation comprising:
an immediate release layer comprising amoxicillin and clavulanate;
a slow release layer comprising amoxicillin and one or more release retarding agents; and,
one or more non-release controlling inert barrier layers placed in between the immediate release layer and the slow release layer and comprising one or more pharmaceutically acceptable excipients.

2. The multilayered modified release formulation according to claim 1, wherein the ratio of amoxicillin and clavulanate is about 16:1.

3. The multilayered modified release formulation according to claim 2, wherein the amount of amoxicillin is 1000 mg±5% and clavulanate is 62.5 mg±5%.

4. The multilayered modified release formulation according to claim 1, wherein the release retarding agent comprises one or more of gums selected from xanthan gum, guar gum, agar, carrageenan, tragacanth, locust bean gum, acacia; and hydrophilic polymers selected from cellulose derivatives, polyvinylalcohol, polyvinylpyrrolidone, polyethylene oxide and alginic acid or salts thereof.

5. The multilayered modified release formulation according to claim 4, wherein the cellulose derivative comprises one or more of carboxymethylcellulose, hydroxypropylcellulose, and hydroxypropyl methylcellulose.

6. The multilayered modified release formulation according to claim 1, wherein the amount of the release retarding agent is 0.5-50% w/w based on the weight of the slow release layer.

7. The multilayered modified release formulation according to claim 1, wherein the immediate release layer and the slow release layer further comprise one or more pharmaceutically acceptable excipients.

8. The multilayered modified release formulation according to claims 1 or 7, wherein the pharmaceutically acceptable excipient comprises one or more of diluents, disintegrants, binders, pH modifiers, lubricants, glidants and coloring agents.

9. The multilayered modified release formulation according to claim 8, wherein the diluent comprises one or more of microcrystalline cellulose, lactose, mannitol and calcium phosphate.

10. The multilayered modified release formulation according to claim 8, wherein the disintegrant comprises one or more of croscarmellose sodium, sodium starch glycolate, crospovidone, hydroxypropyl cellulose, pregelatinised starch and microcrystalline cellulose.

11. The multilayered modified release formulation according to claim 8, wherein the binder comprises one or more of polyvinylpyrrolidone, pregelatinised starch and methacrylic acid polymers.

12. The multilayered modified release formulation according to claim 8, wherein the pH modifier comprises one or more of citric acid, ascorbic acid, tartaric acid, malic acid, malonic acid, succinic acid, fumaric acid, maleic acid, adipic acid, lactic acid, levulinic acid, sorbic acid, polyacrylic acid, orthophosphoric acid, hydrochloric acid, nitric acid, sulphuric acid, sulfamic acid, hydrofluoric acid, oxoacids, sodium carbonate, sodium bicarbonate, magnesium carbonate, magnesium oxide, calcium carbonate, calcium oxide, aluminium hydroxide, magnesium hydroxide, and sodium hydroxide or pharmaceutically acceptable salts thereof.

13. The multilayered modified release formulation according to claim 1, wherein more than 50% of clavulanic acid is released within 15 minutes and more than 40% of amoxicillin is released within 30 minutes, when in vitro release profile is measured using USP-2 method, at 50 rpm, in 900 ml water at 37±0.5° C.

14. A process for the preparation of a multilayered modified release formulation, the process comprising the steps of:
a) preparing immediate release granules comprising amoxicillin, clavulanate and optionally one or more pharmaceutically acceptable excipients; and,
b) preparing non-release controlling inert barrier granules comprising one or more pharmaceutically acceptable excipients; and,
c) preparing slow release granules comprising amoxicillin, one or more release retarding agents and optionally one or more pharmaceutically acceptable excipients; and,
d) compressing the granules of steps (a), (b) and (c) into three separate layers.
